(19) United States
(12) Patent Application Publication
(10) Pub. No.: US 2018/0280622 A1
(43) Pub. Date: Oct. 4, 2018

(54) SYRINGES WITH PLUNGER ROD DOSE CONTROL MECHANISM

(71) Applicant: MEDIMMUNE, LLC, Gaithersburg, MD (US)

(72) Inventors: Bo LI, Gaithersburg, MD (US); Daniel HEINSBERGEN, Gaithersburg, MD (US); Janardhanan Anand SUBRAMONY, Gaithersburg, MD (US)

(73) Assignee: MEDIMMUNE, LLC, Gaithersburg, MD (US)

(21) Appl. No.: 15/766,123

(22) PCT Filed: Oct. 3, 2016
(86) PCT No.: PCT/US16/55129

§ 371 Date: Apr. 5, 2018

Related U.S. Application Data
(60) Provisional application No. 62/238,225, filed on Oct. 7, 2015.

(51) Int. Cl. A61M 5/315

(52) U.S. Cl. CPC A61M 5/31501; A61M 2005/3118; A61M 2005/31508; A61M 5/31536

(57) ABSTRACT

This invention discloses pre-filled syringes and adjustable or movable dose limit fittings that cooperate with the plunger rod to titrate dosage, and methods for using same.

### FIELD OF THE INVENTION

This invention generally relates to syringes and more specifically to pre-filled syringes that have a dose control mechanism cooperating with the plunger rod to control the dose(s) exiting the syringes.

### BACKGROUND OF THE INVENTION

Prefilled syringes (PFS) have been widely used as delivery devices for pharmaceutics, medicines, vaccines, etc. Multiple or partial dosages are often needed for various clinical or treatment purposes or for patients of varying ages and sizes. PFS with different fill volumes are developed and validated to meet the multi dose requirement. However for each fill volume, a separate fill finish process has to be developed and validated, as well a product release process. Multiple stability programs have to be developed and implemented for regulatory filings and product quality control purposes. Current fill finish process can fill as low as 0.15 ml into industry standard PFS when low volume is required; however, the volume accuracy still needs to be confirmed. Low fill volumes can also pose challenges to visual inspection of product release.

A Kineret® PFS achieves multiple dosing by requiring patient to expunge excess drug product and push the PFS plunger to the desired dose. This, however, requires great device handling skill and accurately controlled operation. Unintended over-displacement of the plunger rod would cause insufficient drug product left in the PFS, underdosing, unnecessary product waste and potential compliant issues.

U.S. Pat. No. 5,601,077 to Imbert and related patent(s) disclose a syringe with a removable dose limiter attached to the plunger rod. This dose limiter cannot be adjusted to vary the dosage to be administered.

Hence, there remains a need for improved PFS's that allow more controls over the dosages to be administered.

### SUMMARY OF THE INVENTION

The present invention relates to a universal pre-filled syringe (PFS) which has been filled at the fill site with a set volume of substance, and which has the ability to deliver different injection volumes. A plunger fitting in one embodiment is permanently or removably attached to the PFS' plunger rod. The plunger fitting allows adjustment of the length of the plunger rod at the fill site. The fitting is adjusted on the plunger rod at the fill site to allow only the specified dosage to be delivered. For one substance, different lots of PFS may be set at fill site to deliver different volumes of substance by adjusting the fitting on the plunger rod at the fill site.

In one embodiment, the present invention is directed to a method for using a pre-filled syringe with a dose control fitting disposed on a plunger rod, wherein the dose control fitting limits the movement of the plunger rod into the syringe. This method comprising the steps of:

a. filling syringes to the same volume at the fill site;

b. setting the dose control fitting on the plunger rod to adjust the plunger rod to deliver the desired volume;

c. assembling the pre-filled syringes with the adjusted plunger rods.

In another embodiment, the present invention is directed to a PFS comprising a barrel and a plunger rod containing a volume of liquid between the barrel and the plunger rod. The PFS further comprises a dose control fitting disposed on the plunger rod. The dose control fitting limits the movement of the plunger rod into the barrel to express the liquid from the syringe, and the dose control fitting is set at the fill site to limit the plunger rod movement to deliver the desired volume of liquid.

In one embodiment, the dose control fitting may be a cap that fits over a proximal end of the plunger rod and the cap is movable relative to the plunger rod. The cap comprises at least one spring arm sized and dimensioned to cooperate with at least one notch defined on the plunger rod to fix the relative positions of the cap and the plunger rod. The at least one notch is located on a notched side of the plunger rod and the plunger rod further comprises a smooth side.

In another embodiment, the dose control fitting comprises at least one internal arm that cooperates with a plurality of notches defined on the plunger rod. The relative positions of the dose control fitting cap and the plunger rod are fixed when the at least one internal arm is received in one of the plurality of notches. The plunger rod further comprises a smooth portion such that the at least one internal arm can slide thereon.

In another embodiment, the dose control fitting comprises at least one internal protrusion that cooperates with a plurality of bayonet cut-outs defined on the plunger rod. The relative positions of the dose control fitting and the plunger rod are fixed when the at least one protrusion is received in one of the plurality of bayonet cut-outs. The plunger rod further comprises a smooth portion such that the at least one internal protrusion can slide thereon.

In another embodiment, the dose control fitting comprises a spring biased button that cooperates with a roughened channel. The relative positions of the dose control fitting and the plunger rod are fixed when the spring biased button is received in the roughened channel. The dose control fitting is movable relative to the plunger rod when the spring biased button is moved away from the roughened channel.

In yet another embodiment, the dose control fitting comprises internal threads sized and dimensioned to rotate on external threads on the plunger rod.

In another embodiment, the present invention is directed to a pre-filled syringe (PFS) comprising a barrel and a movable plunger containing a volume of liquid between the barrel and the piston. The PFS further comprises a dose control fitting disposed on a plunger rod connected to the piston. The dose control fitting limits the movement of the plunger rod into the barrel to express the liquid from the syringe, and the length of the dose control fitting is adjustable.

In an embodiment, the invention is directed to a customized prefilled cartridge or device (such as a syringe) with a removable or detachable needle. The cartridge or device comprises a plunger and a plunger rod, and an elastomer seal for attaching a needle. The cartridge or device may be filled with substance useful for administering more than one dose. The plunger comprises a dose control fitting such that after administration of a dose the needle is removed and the cartridge or device may be stored. When another dose is to be administered, a new needle is attached to the cartridge or device, and the plunger is used to expel this dose.

This dose control fitting may comprise telescopic parts and a fastener to lock the positions of the telescopic parts on the plunger rod to determine the length of the plunger rod.

The dose control fitting may comprise two threaded parts connected to each other, wherein the two parts are rotated relative to each other to determine the length of the plunger rod.

The dose control fitting may comprise a plurality of segments connected by weakened webs.

The dose control fitting may comprise a plurality of tear-able tabs attached to a shaft of the plunger rod.

The dose control fitting may comprise a dose limiter of predetermined size.

Another embodiment is directed a pre-filled syringe comprising a barrel, a movable piston containing a volume of liquid between the barrel and the piston, and a plurality of plunger rods adapted to push the piston to express the liquid from the pre-filled syringe. Each plunger rod comprises a dose control fitting thereon and the dose control fittings are located at a different location on the plurality of plunger rods, wherein each dose control fitting limits the movement of the plunger rod into the barrel.

### BRIEF DESCRIPTION OF THE DRAWINGS

In the accompanying drawings, which form a part of the specification and are to be read in conjunction therewith and in which like reference numerals are used to indicate like parts in the various views:

FIGS. 1A and 1B show a conventional syringe with no dose limiter, and FIGS. 1C and 1D show another conventional syringe with a fixed dose limiter;

FIGS. 2-4 show variable dose control fittings that can be fitted around a plunger rod;

FIGS. 5 and 6 illustrate a plunger rod with a dose control fitting positioned at different locations;

FIG. 7 shows a plunger rod with a plurality of frangible or removable dose control fittings;

FIGS. 8A and 8B are perspective views of a telescopic dose control fitting on a plunger rod FIG. 11C is a cross-sectional view of FIG. 11B;

FIG. 9A shows a plunger rod with a sliding dose control fitting and FIG. 9B is a partial exploded cross-section al view of the sliding dose control fitting; and

FIG. 10A shows a plunger rod with another sliding dose control fitting and FIG. 10B is a partial exploded view of the sliding dose control fitting;

FIG. 11A shows a plunger rod with another sliding dose control fitting and FIG. 11B is a cross-sectional view of the plunger rod shown in FIG. 11A; and

FIG. 12 shows a plunger rod with a rotating, sliding dose control fitting.

### DETAILED DESCRIPTION OF THE PREFERRED EMBODIMENTS

The present invention allows for PFS to be filled to the same volume at the fill site, and to be assembled with a plunger rod containing a dose control fitting set (installed or placed) to the desired dosage volume. This extends the advantages of prefilling syringes with pharmaceutics. Only a single fill finish and product release process needs to be developed and validated for each fill volume of a specific PFS/product combination, and only a single stability program needs to be developed and implemented for regulatory filings and product quality control purpose.

The present invention also allows accurate low volume dosage at the point of use. Accuracy of low dosage volumes, which can range as low as 0.15 ml; as low as 0.20 ml; up to and including at least to 1.0 ml, or at least 1.5 ml, at the point of injection can be difficult to maintain. Syringes contain volumes that retain liquids even after the plunger is fully depressed into the barrel of the syringes. The un-usable or un-injectable volumes include the volume inside the hypodermic needle, the hollow connector on the syringe that receives the needle (luer lock or slip on), and the space, if any, between the distal end of the barrel and the distal end of the plunger. For low dosage volumes, this unusable or un-injectable volume must be calculated or compensated. The un-injectable volume may be in the same order of magnitude as the low dosage volume, and any air or void spaces in this volume could cause an incorrect dosage unless they are primed with liquid. Furthermore, the measurements of small volumes can be challenging when the standard equipment used has significantly larger volumes or when the equipment has poor volume sensitivity. Standard filling equipment may not be able to provide satisfactory accuracy for low volume fillings.

The present invention is also directed to a dose adjustable PFS with removable/attachable needle that may act as a low cost daily injection device as an alternative to expensive daily injection pens. A customized prefilled cartridge or device (such as a syringe) with a removable or detachable needle is envisioned. The cartridge or device comprises a plunger and a plunger rod, and an elastomer seal for attaching a needle. The cartridge or device may be filled with substance useful for administering more than one dose. The plunger comprises a dose control fitting such that after administration of a dose the needle is removed and the cartridge or device may be stored. When another dose is to be administered, a new needle is attached to the cartridge or device, and the plunger is used to expel this dose. This will allow ease of transportation/portability of the PFS, use less storage space (such as in the refrigerator), and have less moving parts such as spring.

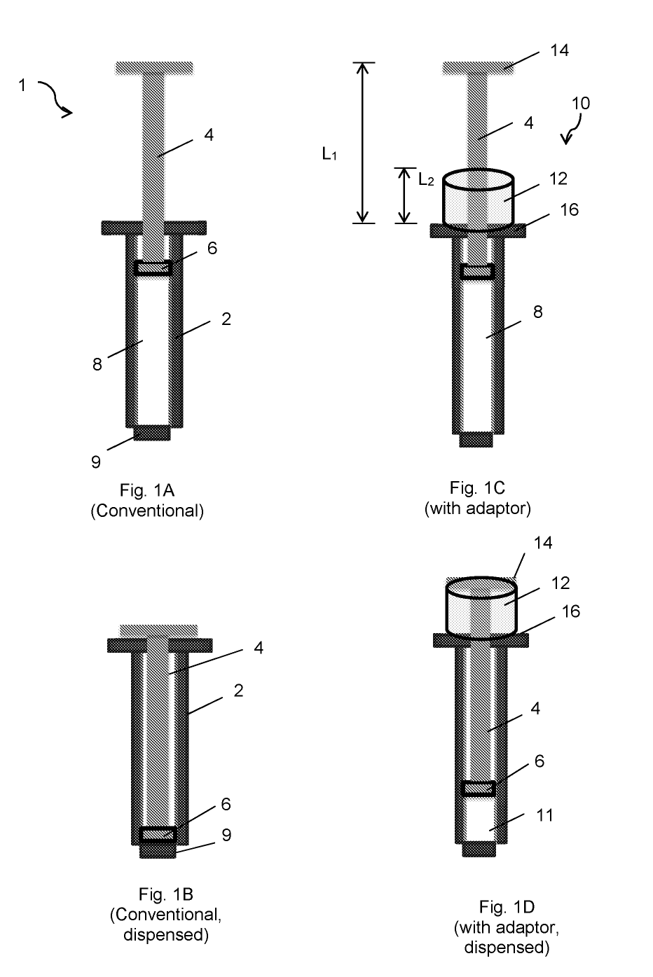

Referring to FIGS. 1A and 1B, a conventional PFS 1 is shown with a barrel 2 and plunger rod 4. Plunger rod 4 has a rubber tip or piston 6 at this distal end, and barrel 2 is sized and dimensioned to hold a fixed liquid volume 8 of pharmaceutic, medicine, or vaccine. When plunger rod 4 is depressed, the liquid is pushed out from PFS 1 at exit 9 through a hypodermic needle, not shown. FIGS. 1C and 1D, which illustrate another conventional PFS, show a PFS with a fitting 12 disposed around plunger rod 4 or between head 14 of plunger rod 4 and flange 16 of barrel 6. L₁ is the distance between flange 16 and head 14, or the full distance that plunger rod 4 may travel when fitting 12 is not present. L₂ is the length of fitting 12 and (L₁−L₂) is the distance that plunger rod 4 may travel with fitting 12 in place. The fitting may be different size, depending on the volume or dosage intended to be delivered. A fitting of specified size is assembled on to the plunger rod at fill site to deliver desired dosage to patient.

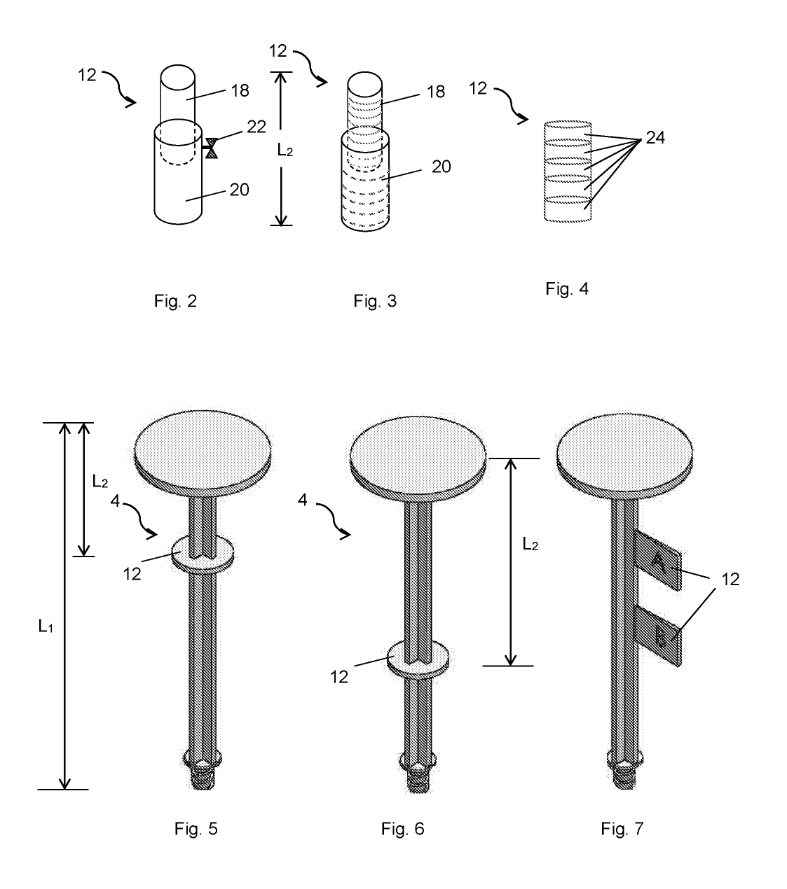

FIGS. 2-4 illustrate variable dosage fittings 12 of the present invention. FIG. 2 shows two telescopic halves 18 and 20 of fitting 12. One half fits inside the other half and the length L₂ of fitting 12 is fixed by screw 22 or the like. Halves 18 and 20 are positioned so that length L₂ is set, and then screw 22 is tightened to fix this length. To change this length, screw 22 is loosen and the halves 18 and 20 are moved telescopically relative to each other before screw 22 is re-tightened to select another length. Once the length of the dosage fitting is determined, the PFS is assembled with the dosage fitting at the desired length.

FIG. 3 shows variable dosage fitting 12 with half 18 with external threads that are sized and dimensioned to fit within the internal threads on half 20. The two halves 18 and 20 are rotated relative to each other to select the desired length L₂. FIG. 4 shows dosage fitting 12 comprising a plurality of segments 24 lightly connected to each other by tear-away webs. One or more segments 24 can be torn away to select a desired length L₂ for dosage fitting 12.

FIGS. 5 and 6 illustrate another embodiment of plunger rod 4, which has a dosage fitting 12 made integral thereto. In this case, a plurality of rods 4 with fitting 12 located at different locations along its shaft are available at fill site and once the dosage is determined, the appropriate dosage fitting is assembled with PFS 10. Alternatively, the plunger rod may be assembled at the point of use preferably by medical personnel. The dosage volume 11 in PFS 10 is determined by the location of fitting 12 on the shaft of plunger rod 4. FIG. 7 shows a plunger rod 4 with multiple fitting tabs 12. While two fitting tabs are shown, any number of tabs can be employed. One or more tabs are pulled or torn off plunger rod 4 to determine the dosage volume 11 prior to assembly at fill site.

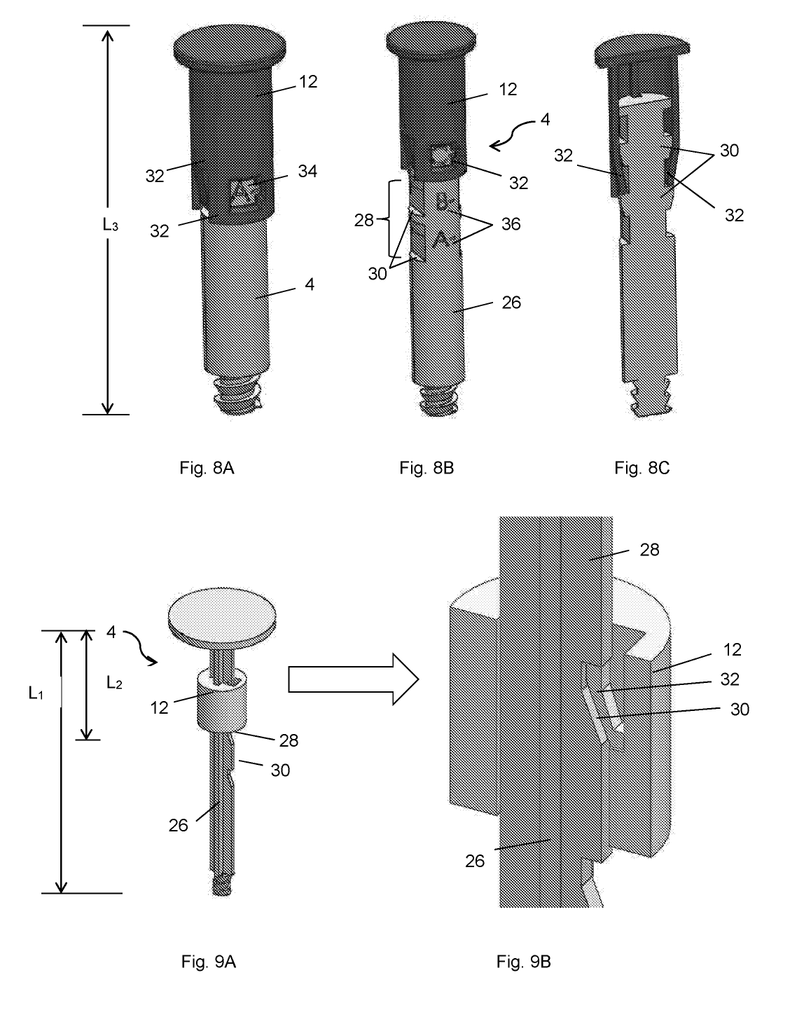

In the embodiment shown in FIGS. 8A-8C, dosage fitting 12 is a cap fitted over the top of, and on the outside of plunger rod 4. As best shown in FIG. 8B, plunger rod 4 has a smooth side 26 and notched side 28 comprising a plurality of notches 30. Fitting 12 has at least one spring arm 32 that is biased inward so that the distal tip of spring arm 32 would come into contact with notch 30 to arrest relative motion between fitting 12 and plunger rod 4, i.e., to arrest the movement of fitting 12 and plunger rod 4 toward each other as best shown in FIG. 8C. Preferably, two spring arms 32 are used; however, any number can be used. To change the combined length L₃, fitting 12 is rotated so that spring arms 32 can slide on smooth side 26. Preferably, a window 34 is provided on a spring arm so that indicia 36, e.g., A, B, C indicating the volume 11, written on plunger arm 4 can be seen through window 34. When a desired dosage volume 11 as indicated by indicia 36 is seen, fitting 12 is rotated to that spring arms 32 are located on notched side 28 and engage notches 30. The combined length L₃ of plunger rod 4 and fitting 12 is now locked or set and the plunger rod may be assembled with the PFS. Longer combined length L₃ corresponds with lower dosage volume 11. In a variation of this embodiment, smooth side 26 is omitted and notched side 28 extends around plunger rod 4. Fitting 12 is preferably pulled away from plunger rod 4 to set the combined length L or to set the dosage volume 11 of the PFS. In this embodiment, the total length L₃ of plunger rod 4 is effectively varied.

In the embodiment shown in FIGS. 9A and 9B, fitting 12 is slidable along the shaft of plunger rod 4. Plunger rod 4 also has a smooth side or rail 26 and notched rail or side 28. Fitting 12 has an internal arm 32 that points toward plunger rod 4. To slide up and down plunger rod 4, fitting 12 is rotated until arm 32 rides on smooth rail 26 or on the corner between notched rail 28 and smooth rail 26. To lock into position and a desired effective length (L₁−L₂) as illustrated in FIG. 9A, fitting 12 is rotated to that its arm 32 is received within notch 30 on notched rail 28. To adjust the effective length (L₁−L₂), fitting 12 is rotated so that arm 32 is away from notch 30. A new length is selected and fitting 12 is rotated back until arm 32 is received in another notch 30.

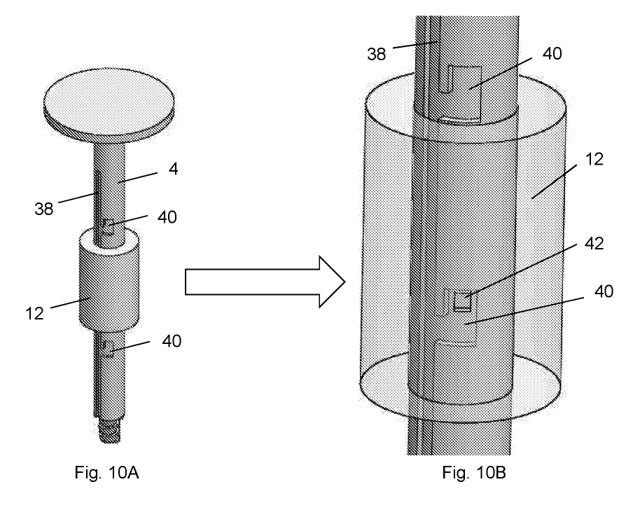

Another embodiment showing a sliding fitting 12 is shown in FIGS. 10A and 10B. Here, plunger rod 4 has an indented rail 38, which is preferably smooth, defined on its surface. The surface of plunger rod 4 also has a plurality of bayonet cut-outs 40 connected to indented rail 38 as shown. Fitting 12 has an internal protrusion 42, which is sized and dimensioned to slide or ride freely within indented rail 38 and to be received in cut-outs 40. To lock the travel length of plunger rod 4 to determine the dosage volume 11, fitting 12 is aligned so that its protrusion 42 can enter a bayonet cut-out 40 and is held therewithin, as shown in FIG. 10B. In a bayonet connection, protrusion 42 is rotated to enter cut-out 40 at its opening, then fitting 12 is moved translationally to lock protrusion 42 in a closed end of the cut-out away from the opening. The location or position of fitting 12 can be changed by reversing the locking process, moving fitting 12 to align with another cut-out 40 and executing the locking process again.

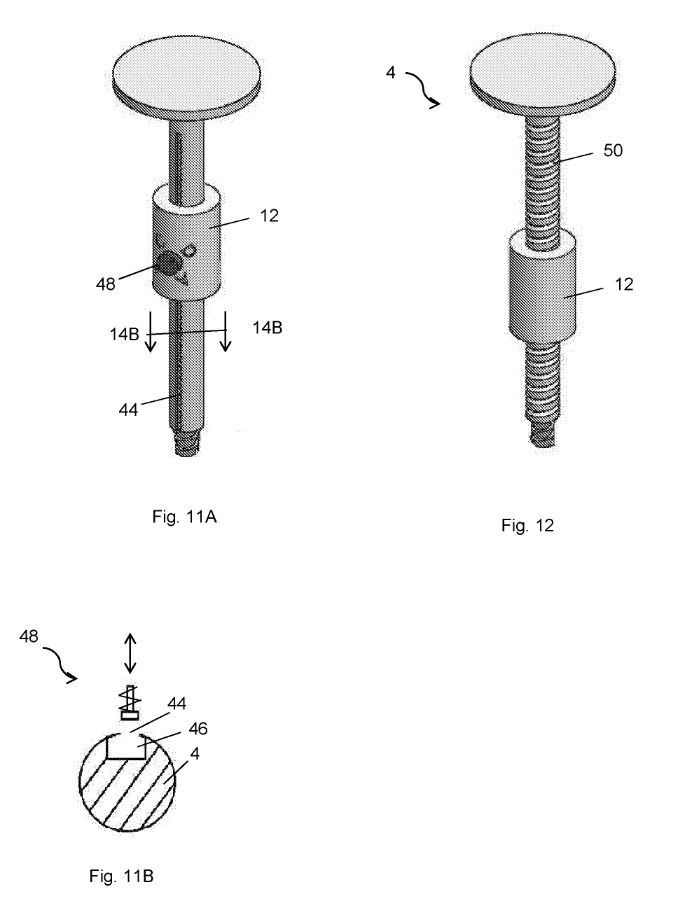

Another sliding dosage fitting 12 is shown in FIG. 11A-11B. Here plunger rod 4 has a roughened or toothed rail 44. Tooth rail 44 may be connected to an enlarged hollow cavity 46 located immediately below it. Dosage fitting 12 has an internal spring-biased toothed protrusion 48, which is biased to engage toothed rail 44. When engaged, the location of fitting 12 on plunger rod 4 is fixed. To move fitting 12, protrusion 48 is pushed into enlarged cavity 46 or is pulled into fitting 12 to disengage toothed protrusion 48 from toothed rail 44. Fitting 12 is moved to another position and then the force on protrusion 48 is removed thereby allowing toothed protrusion to re-engage with toothed rail 44.

Another movable fitting 12 is illustrated in FIG. 12. In this embodiment, the shaft of plunger rod 4 has external threads 50 which are designed to threadedly engage with the internal threads of dosage fitting 12. As dosage fitting 12 is rotated around plunger rod 4, dosage fitting 12 can move up and down plunger rod 4 to determine how far the plunger rod 4 travels into the PFS thereby determining the dosage volume 11.

While it is apparent that the illustrative embodiments of the invention disclosed herein fulfill the objectives stated above, it is appreciated that numerous modifications and other embodiments may be devised by those skilled in the art. Therefore, it will be understood that the appended claims are intended to cover all such modifications and embodiments, which would come within the spirit and scope of the present invention.

1. A method for using a prefilled syringe with a dose control fitting disposed on a plunger rod, wherein the dose control fitting limits the movement of the plunger rod into the syringe, said method comprising the steps of:
a. filling syringes to the same volume at fill site;
b. setting a dose control fitting on to the plunger rod to adjust the plunger rod to deliver the desire dosage;
c. assembling pre-filled syringes with the adjustable plunger rods to deliver different dosages.

2. A pre-filled syringe containing a set volume, and assembled with a plunger rod comprising a dose control fitting set to dispose a different volume depending on the configuration of the dose control fitting.

3. A pre-filled syringe comprising a barrel and a movable piston containing a volume of liquid between the barrel and the piston, wherein a plunger rod with a dose control fitting disposed on the plunger rod is adapted to push the piston to express the liquid from the pre-filled syringe, wherein the dose control fitting limits the movement of the plunger rod into the barrel.

4. The pre-filled syringe of claim 3, wherein the dose control fitting comprises a cap that fits over a proximal end of the plunger rod and the cap is movable relative to the plunger rod.

5. The pre-filled syringe of claim 4, wherein the cap comprises at least one spring arm sized and dimensioned to cooperate with at least one notch defined on the plunger rod to fix the relative positions of the cap and the plunger rod.

6. The pre-filled syringe of claim 5, wherein the at least one notch is located on a notched side of the plunger rod and the plunger rod further comprises a smooth side.

7. The pre-filled syringe of claim 3, wherein the dose control fitting comprises at least one internal arm that cooperates with a plurality of notches defined on the plunger rod, wherein the relative positions of the dose control fitting cap and the plunger rod are fixed when the at least one internal arm is received in one of the plurality of notches.

8. The pre-filled syringe of claim 7, wherein the plunger rod further comprises a smooth portion such that the at least one internal arm can slide thereon.

9. The pre-filled syringe of claim 3, wherein the dose control fitting comprises at least one internal protrusion that cooperates with a plurality of bayonet cut-outs defined on the plunger rod, wherein the relative positions of the dose control fitting and the plunger rod are fixed when the at least one protrusion is received in one of the plurality of bayonet cut-outs.

10. The pre-filled syringe of claim 9, wherein the plunger rod further comprises a smooth portion such that the at least one internal protrusion can slide thereon.

11. The pre-filled syringe of claim 3, wherein the dose control fitting comprises a spring biased button that cooperates with a roughened channel, wherein the relative positions of the dose control fitting and the plunger rod are fixed when the spring biased button is received in the roughened channel.

12. The pre-filled syringe of claim 11, wherein the dose control fitting is movable relative to the plunger rod when the spring biased button is moved away from the roughened channel.

13. The pre-filled syringe of claim 3, the dose control fitting comprises internal threads sized and dimensioned to rotate on external threads on the plunger rod.

14. The pre-filled syringe of claim 3, wherein a length of the dose control fitting is adjustable.

15. The pre-filled syringe of claim 14, wherein the dose control fitting comprises two telescopic parts and a fastener to lock the positions of the two parts relative to each other to determine said length.

16. The pre-filled syringe of claim 14, wherein the dose control fitting comprises two parts threadedly connected to each other, wherein the two parts are rotated relative to each other to determine said length.

17. The pre-filled syringe of claim 14, wherein the dose control fitting comprises a plurality of segments connected by weakened webs.

18. The pre-filled syringe of claim 14, wherein the pre-filled syringe is assembled at the fill site with a plunger rod adapted to express the desired volume.

19. The pre-filled syringe of claim 3, wherein the dose control fitting is slidable relative to the plunger rod.
